(19) United States
(12) Patent Application Publication
(10) Pub. No.: US 2012/0301532 A1
(43) Pub. Date: Nov. 29, 2012

(54) COMPOSITIONS AND METHODS FOR TREATING MATERIALS WITH INSECTICIDES AND POTENTIATING AGENTS

(75) Inventors: H. Augustus Carey, Ambler, PA (US); James B. Ballard, Medford, NJ (US); Joseph S. Latino, Brooklyn, NY (US)

(73) Assignee: ALLERGY TECHNOLOGIES, LLC, Ambler, PA (US)

(21) Appl. No.: 13/482,931

(22) Filed: May 29, 2012

Related U.S. Application Data
(60) Provisional application No. 61/490,206, filed on May 26, 2011.

(51) Int. Cl. A01N 63/02; A01N 47/40; A01N 53/08; C07C 47/21; A01P 19/00; A01P 7/04; A01N 35/02; A01N 25/08; A01N 35/04

(52) U.S. Cl. 424/411; 424/84; 568/448

(57) ABSTRACT

In accordance with the present disclosure, compositions and methods are described for potentiating the effectiveness of insecticides with which materials or objects are treated or impregnated to reduce or eliminate insect populations that infest or inhabit materials, articles and furnishings associated with human or animal use. Materials or articles may be treated or impregnated with compositions comprising one or more insecticides in combination with one or more potentiating agents that effectively promote the movement or aggregation of insects to a designated area that has been treated or impregnated with insecticide, and delivery devices containing a potentiating agent or agents may be associated with a material or article that has been previously treated or impregnated with insecticide.

### CROSS-REFERENCE TO RELATED APPLICATIONS

This is a non-provisional application which claims priority to U.S. Ser. No. 61/490,206, filed May 26, 2011, the entire contents of which are incorporated herein.

### STATEMENT REGARDING FEDERALLY SPONSORED RESEARCH OR DEVELOPMENT

None.

### REFERENCE TO SEQUENCE LISTING, A TABLE, OR A COMPUTER PROGRAM LISTING COMPACT DISC APPENDIX

None.

### BACKGROUND OF THE INVENTIONS

1. Field of the Inventions

This disclosure relates to the treatment of materials with insecticidal compositions to reduce insect populations, and particularly relates to treatment of materials for use in commercial and residential furnishings to effect an enhanced reduction in insect populations in furnishings.

2. Background of Related Art

The incidence of bed bug infestations in both commercial and residential settings has increased significantly over the last few years. Certain species of bed bugs feed on human and animal blood, and inflict painful and injurious bites that may be contributory to other disease conditions, such as bacterial and other pathogenic infections. Additionally, other insects are known to infest or invade human and animal habitats and cause enumerable injuries or health conditions, including bites or stings, rashes, infections and allergies. Serious secondary infections that result from scratching the primary site of the insect bite have been documented, especially in the young, elderly and those with compromised immune systems.

The problem of insect infestation in human and animal habitats has been addressed in various ways. For example, U.S. Pat. No. 6,440,438 discloses methods of impregnating textiles, particularly voile, with the insecticide permethrin, and discloses the use of such impregnated textiles for use as bed clothing and fabric coverings for other furnishings such as chairs and couches. U.S. Application No. 2010/0183690 discloses a similar fabric impregnation methodology which involves the microencapsulation of an insecticide to provide extended release of the insecticide into bed clothing and the like.

### BRIEF SUMMARY OF THE INVENTIONS OF THE DISCLOSURE

In accordance with the present disclosure, compositions and methods are described for potentiating the effectiveness of insecticides, with which materials or objects are treated or impregnated, to reduce or eliminate insect populations that infest or inhabit objects and furnishings associated with human or animal use.

In one aspect of the disclosure, materials or objects are treated or impregnated with one or more insecticides, and are further treated with a potentiating agent that effectively promotes the movement or aggregation of insects to a designated area that has been treated or impregnated with insecticide. “Potentiating agents,” as used herein, are compounds or compositions that have the effect of attracting or drawing insects to a site that has been treated with one or more insecticides to increase the rate of contact between the insect and the locus of insecticide.

In one aspect of the disclosure, potentiating agents are made of natural and/or synthetically-produced compounds or compositions that act to attract, draw or lure insects toward a source or locus of insecticide with which a material or object has been treated or impregnated. Compounds and compositions that may be used as potentiating agents include one or more natural semiochemicals, or similar natural and/or synthetically-produced compounds in an amount sufficient to cause an attraction of insects to a source or locus of insecticide that resides in products associated with human and animal habitation. “Semiochemicals,” as used herein, refers to compounds that act as message-bearing molecules that are used by an organism as signals. Unlike visual or auditory signals, semiochemicals are volatile and are perceived through olfaction. The messages that semiochemicals convey to insects result in specific behaviors, and can be used as tools for integrated pest management because of their ability to modify organism behavior.

Certain semiochemicals are naturally occurring and are not toxic. They have a high degree of species specificity and low environmental impact. Pheromones are a subset of the larger, more diverse group of semiochemicals. The term “pheromone,” as used herein, applies to chemical signals that are emitted and perceived within the same or closely related species. Pheromones that are useful in the present invention are not limited to attractants, since anti-aggregation, arrestant, oviposition deterrents and alarm pheromones are also useful types of pheromones that may be employed as potentiating agents in certain concentrations.

As used herein, “semiochemical-like” refers to compounds or compositions that are, or contain, synthetically-produced compounds that act like, or in a manner similar to, naturally-occurring semiochemicals to attract, draw or lure insects to a site. Semiochemical-like compounds or compositions may be comprised of all synthetically-produced compounds, or may be a mixture of natural and synthetically-produced compounds.

In another aspect of the disclosure, methods are employed for treating or impregnating materials or objects with one or more insecticides, and then treating such materials or objects with one or more potentiating agents to enhance or increase the parameters of exposure or contact between the insects and the insecticide.

In yet another aspect of the disclosure, methods are employed for treating or impregnating materials or objects with a composition comprising one or more insecticidal agents and one or more potentiating agents in admixture.

Methods of the present disclosure are beneficially carried out on materials and objects associated with human and animal habitation, including home, office and school furnishings, linens, clothing, toys, areas of public transportation and numerous other objects and furnishings of everyday life in connection with which insects may infest and cohabitate.

In yet another aspect of the disclosure, materials or objects that have been previously treated or impregnated with one or more insecticides are further treated with a potentiating agent, in accordance with the present disclosure, to increase the effectiveness of the one or more previously applied insecticides to reduce or eliminate insect populations in or on materials and objects associated with human and animal habitation. In accordance with this aspect of the disclosure, potentiating agents are provided in delivery systems that are associated with materials or objects that have been previously treated with one or more insecticides.

In still another aspect of the disclosure, delivery systems are provided for treating materials or objects, which have been treated or impregnated with one or more insecticides, with one or more potentiating agents to enhance the efficacy of the insecticide.

In yet another aspect of the disclosure, materials or objects are provided which are treated or impregnated with one or more insecticides, and are also treated with one or more potentiating agents that enhance or increase the efficacy of the one or more insecticides with which material or objects have been treated to effectively potentiate the efficacy of the insecticide in reducing insect populations in or on the material or objects.

### BRIEF DESCRIPTION OF THE SEVERAL VIEWS OF THE DRAWINGS

In the drawings, which depict what is currently considered to be the best modes for carrying out the various embodiments of the present disclosure:

FIG. 1 illustrates schematically one aspect of the present disclosure embodying a material that has been treated with one or more insecticides and one or more potentiating agents; and

FIG. 2 illustrates one exemplar delivery means for treating a material or object with a potentiating agent in accordance with one aspect of the present disclosure.

### DETAILED DESCRIPTION OF THE EMBODIMENTS OF THE DISCLOSURE

The compositions and methods of the present disclosure may be adapted for use in reducing or eliminating infestations of any number of organisms, including a variety of insects, in material or objects that are commonly used in human and animal habitation. However, for ease of explanation, the various aspects and embodiments of the present disclosure may be particularly described herein with respect to treatment for reducing bed bug populations, as one illustrative application or use.

In accordance with a first aspect of the present disclosure, potentiating agents are formulated for use in treating material and objects that are also treated or impregnated with one or more insecticides which are specifically selected to be noxious or toxic to a given species or group of organisms, such as insects. In particular, potentiating agents of the present disclosure are semiochemicals or semiochemical-like compounds or compositions that are capable of drawing or luring a given organism to a site of insecticidal treatment or impregnation.

Bed bugs, for example, are attracted to a host by cues, such as body heat of the host, odors emitted by the host and CO₂. Such attractants bring the bed bugs to the bed and to the food source, i.e., blood. These attractants, however, have a determined range of effectiveness as an attractant. For example, body heat and human body odors have an effective range of about four centimeters or less, while CO₂ emissions have an effective attractant range of about 86 centimeters or more. Insect pheromones or similar semiochemicals have demonstrated effectiveness as attractants, and are beneficial in producing the same attractant effect, but with increased attractant results.

Two principle pheromones may be particularly useful in the aspects of the present disclosure: aggregation pheromones and alarm pheromones. Aggregation pheromones are produced by insects, particularly bed bug species, and have the effect of attracting insects to a locus of emission of the pheromone. Bed bugs, for example, are known to aggregate together in a mattress after a blood feeding due to the emission of aggregation pheromones. Other attractants that may cause this aggregation phenomenon are the presence of eggs, egg cases, molted skin (casts), feces of the bed bug and blood.

Aggregation pheromones have been analyzed from the volatiles emitted from bed bugs in closed containers. These aggregation pheromones include (E)-2-hexenal and (E)-2-octenal (most plentiful and produced by adult bed bugs), 4-oxo-(E)-2-hexenal and 4-oxo-(E)-2-octenal (produced by nymph bed bugs), and also includes nonanal, decanal, (2E,4E)-octadienal, benzaldehyde, (+)- and (−)-limonene, sulcatone, benzyl alcohol and a myriad of other entities of various volatilities. This assemblage of naturally occurring chemicals, also referred to as the “volatile blend” or aggregation pheromones has a demonstrated effective attractant range of greater than thirty centimeters. Each constituent of this volatile blend of aggregation pheromones can be synthetically derived, and may also be referred to as semiochemical-like compounds.

Another effective component for compositions of the present disclosure are solvent extracts of paper exposed to bed bugs for a period of time, which results in a series of attractants which may not all be highly volatile. Those lesser and non-volatile aggregation pheromones are present in the feces and cuticular secretions of bed bugs. Paper exposed to bed bugs, also referred to herein as “conditioned paper” or “conditioned substrate,” is used in the making of the solvent extracts. A fabric or paper substrate (e.g., filter paper) having a surface area of approximately 10 to 100 cm² is exposed to between five and twenty-five mixed sex adult bed bugs. Exposure of the bed bugs to the substrate may last from between five to thirty days, with a common period of exposure ranging from ten to twenty days. Following the period of exposure, all bed bugs, nymphs and eggs are removed from the conditioned substrate before being used further.

In alternative aspects of the disclosure, conditioned substrates may be produced by exposing fabric or paper to a mix of ten to fifty mixed-sex adult bed bugs and ten to fifty assorted nymph bed bugs. Following the period of exposure, all bed bugs, nymphs and eggs are removed from the conditioned substrate before being used further. The conditioned substrates described can be used as the source of potentiating agents in carrying out the methods of the present disclosure, as described more fully hereinafter.

In another aspect of the disclosure, the conditioned substrate may be extracted in an organic solvent such as, for example, methanol or acetone, and allowed to air dry. The dried component may then be used as the potentiating agent. The dried component has, most suitably, an effective attractant range of at least 30 cm away from the source of the potentiating agent. The combination of the volatile blend plus the solvent extract blend is thought to have an attraction range of more than 30 cm.

Other effective types of pheromone for use in the compositions of the present invention are “alarm” pheromones that are produced by insects, especially bed bugs. Bed bugs produce alarm pheromones when disturbed. Surprisingly, at high levels of the alarm pheromone, the bed bugs disperse; but at low levels, alarm pheromones serve to cause aggregation of the bed bugs. Two exemplary pheromones are volatile alarm pheromones in the aldehyde family: (E)-2-hexenal and (E)-2-octenal. Compositions in accordance with the aspects of this disclosure may include one or more of these compounds. However, each compound is in a final concentration of less than 6×10¹⁵ molecules/ml (1.0×10⁻¹¹ M) for hexenal and 9×10¹⁴ molecules/ml (1.5×10⁻² M) for octenal, which are the upper level concentrations at or below which aggregation is produced by these alarm pheromones. The above-described pheromones may be formulated into compositions that have a selected effective attractant range, preferably in excess of 30 centimeters.

In another aspect of the present disclosure, the compositions of the present invention may comprise or include synthetically-produced compounds, or semiochemical-like compounds that demonstrate the same attractant effectiveness for aggregation of insects as demonstrated by naturally-occurring pheromones. For example, potentiating agents can be constructed from a single synthetic semiochemical, or combination thereof, and may be of an initial concentration range of about 1×10⁻⁶ M to 1×10⁻² M, but most generally in the range of 1×10⁻⁴ M. For example, 100 μl of 1×10⁻⁴ M (E)-2-hexenal in acetone can be added to a 16 cm² fabric or paper substrate and allowed to dry. As a further example, 100 μl of 1×10⁻⁴ M (E)-2-octenal in acetone can be added to a 16 cm² fabric or paper substrate and allowed to dry. Alternatively, and by way of example, 100 μl of a 75/25 mixture of 1×10⁻⁴ M hexanal and octenal, respectively, in acetone can be added to a 16 cm² fabric or paper substrate and allowed to dry. Each treated fabric or paper thus produced is then placed into a delivery system such that the final concentration(s) exposed to the bed bug are less than approximately 6×10¹⁵ molecules/ml (1.0×10⁻¹¹ M) for hexenal and approximately 9×10¹⁴ molecules/ml (1.5×10⁻¹² M) for octenal, respectively, which are the upper level concentrations at or below which aggregation is produced by these alarm pheromones. Compositions in accordance with aspects of the disclosure may be combinations of both natural semiochemicals and synthetically-produced semiochemical-like compounds.

It is a further aspect of the present disclosure that the potentiating agents are placed in proximity to a source of insecticide that is associated by some means with items used normally in the habitation of humans and animals, such as household goods, office or school products and pet products. This may generally be accomplished by positioning a delivery system containing one or more potentiating agents in proximity to or with a source of insecticide, such as may be impregnated in a fabric from which household products are made (e.g., bed linens, furniture coverings, etc.).

The potentiating agents of the present disclosure may be provided in any number of delivery systems that will expose or dispense the potentiating agents slowly into the environment. Delivery systems that encapsulate or house volatile blends of semiochemicals must be able to reduce the degree of volatility by six to eight orders of magnitude in order for the potentiating agent(s) to have a sufficient half-life, as well as to limit the exposure concentration of many of the semiochemicals that otherwise, at higher concentrations, will act as alarm rather than attractant pheromones.

For example, suitable delivery systems may include a carrier device, such as textile patches that are treated or impregnated with one or more potentiating agents, the carrier device, or textile patch, then being suitably sized and structured for attachment to a material or object that has been treated with an insecticide. In addition, conditioned substrates, such as those previously described, may be rolled or otherwise inserted into a perforated cartridge or a plastic ampoule with a semi-permeable membrane, thereby effecting slow release of the volatiles and causing attraction of bed bugs to the insecticide treated fabric.

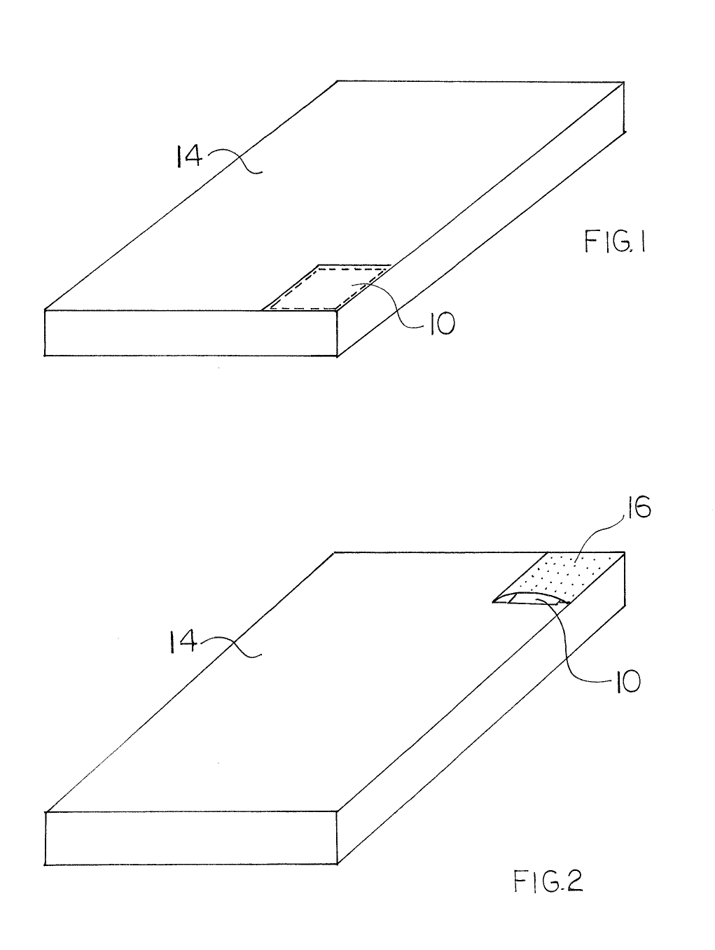

For example, as depicted schematically in FIG. 1, a carrier device 10, depicted as a textile patch, may be stitched directly to an insecticide-impregnated material 14 (e.g., a mattress cover). Alternatively, as depicted in FIG. 2, the carrier device 10 may be removably positioned within a pervious enclosure 16 or housing, depicted, for example, as a perforate envelope, which is secured to an insecticide-impregnated material 14. In the latter type of delivery mode, as depicted in FIG. 2, the carrier device 10 may be removed and replaced from time-to-time to provide a renewed source of potentiating agent.

In alternative embodiments of the present disclosure, the potentiating agent may be provided in a delivery vehicle, such as an ampoule of plastic that is structured to diffuse the potentiating agents. Various tapes and other plastic type substrates, or microencapsulated vehicular approaches are also suitable delivery systems. The delivery systems of the present disclosure may be attached to an insecticide-impregnated material or object, such as a mattress liner or a sofa covering, much like a label attached to one or more locations on the edge of material or an object of furniture. The delivery systems may also be incorporated, for example, into an elastic edge of a sheet or mattress liner, or can be clipped or attached to the edge of the material or object. Many other similar forms of attachment are within the scope of the disclosure.

Bed bugs are attracted to a host by cues, such as body heat of the host, odors emitted by the host and CO2. Presently, permethrin impregnated mattress liners, are designed for use on mattresses and box springs, and cause reduction in bed bugs and other insects through contact between the insect and the insecticide, with ensuing neurotoxic effects leading to death. However, attraction of the bed bug to the impregnated cover is a passive and statistically happenstance event, typically driven by the bed bug's natural tendency to seek harborage close to the host, but far enough away to remain undisturbed. The use of a potentiating agent may draw the bed bug away from its naturally attracting source, the human host, toward the attached impregnated liner, enhancing the rate and duration of contact between the bed bug and the impregnated fabric, thereby enhancing its insecticidal action.

Use of permethrin by humans, including direct contact with impregnated fabrics, has generated a formidable safety profile of biocompatible use. Extension of this concept for impregnated fabrics, using natural or synthetically-derived pyrethroids, neonicotinoids, other insecticides such as chlorfenapyr and chlorantraniliprole, may be benefitted by the use of a potentiating agent insofar as allowing for a lower concentration of the insecticide in the impregnated fabric while maintaining its efficacy and enhancing contact biocompatibility.

In another aspect of the present disclosure, compositions are provided for treating or impregnating material or objects comprising one or more insecticides and one or more potentiating agents in accordance with described embodiments of this disclosure. That is, compositions are provided which comprise one or more insecticides, that are known to be effective, non-toxic and safe for use in treating articles of human and animal habitation and which are selected to effectively reduce or eliminate a given organism or group of organisms, in admixture with one or more potentiating agents that comprise semiochemicals and/or semiochemical-like compounds or compositions.

Compositions suitable for use in impregnating fabrics in accordance with the invention may be those that include one or more effective insecticides including, but not limited to, pyrethroids, synthetic or naturally occurring, such as permethrin, bifenthrin and deltamethrin, and neonicotinoids, such as acetamiprid, imidacloprid and thiamethoxam, other insecticides such as chlorfenapyr and chlorantraniliprole, and bioeffective stereoisomers of any such compounds.

The compositions of the disclosure may further comprise insecticidal compounds or materials that are formulated from natural ingredients of a type that are not subject to governmental regulation. These exempt ingredients include: castor oil, linseed oil, cedar oil, malic acid, cinnamon and cinnamon oil, mint and mint oil, citric acid, peppermint and peppermint oil, citronella and citronella oil, 2-phenethyl propionate (2-phenylethyl propionate), cloves and clove oil, potassium sorbate, corn gluten meal, putrescent whole egg solids, corn oil, rosemary and rosemary oil, cottonseed oil, sesame (includes ground sesame plant) and sesame oil, dried blood, sodium chloride (common salt), eugenol, sodium lauryl sulfate, garlic and garlic oil, soybean oil, geraniol, thyme and thyme oil, geranium oil, white pepper, lauryl sulfate, zinc metal strips (consisting solely of zinc metal and impurities), and lemongrass oil.

The compositions of the disclosure may further be formulated with one or more surfactants and/or binding agents (e.g., U.S. Pat. No. 7,625,411) or impregnation technologies [i.e., microencapsulated permethrin (e.g., U.S. Pat. No. 7,887,826), nanoparticle (e.g., U.S. Pat. No. 7,288,267), etc.] which may change the activity profile of the materials.

Insects that may be targeted or effected by the compositions of the present disclosure include, but are not limited to, the common bed bug (Cimex lectularius), tropical bed bugs (Cimex hermipterus), cat fleas (Ctenocephalides felis felis), dog fleas (Ctenocephalides canis), deer ticks (Ixodes ricinis), dust mites (Dermataphagoides farinae), cockroaches, silverfish and ants. The compositions are also directed to targeting or affecting the spread or infestation of other potential disease vectors such as body lice (Pediculus humanus) and head lice (Pediculus capitis).

Other insects that may be targeted by use of the compositions and treated textiles, fabrics or materials include mosquitoes which carry various diseases, the species including Anopheles stephensi, Anopheles gambiae, Anopheles albimanus, Culex quinquefasciatus and Ades aegypti. Many flying insects that may be targeted include sandflies (Phlebotomus papatasi), kissing bugs (Rhodnius prolixus), biting midges (Culicoides nebeculosis), house flies (Musca domestica), wasps (Apocrita) and yellow jackets (Vespula vulgaris). Other troublesome insects may include stink bugs, spiders, centipedes, foliage-feeding caterpillars, earwigs, moths and scorpions.

The compositions of the disclosure may be impregnated into a selected textile, fabric or material by methods such as dipping or immersing the textile, fabric or material in a vessel containing the composition. The textile, fabric or material is immersed in the composition for a period of time sufficient to assure that the composition is incorporated onto the fibers of the textile, fabric or material. The impregnated textile, fabric or material is then allowed to dry, and may be dried in the presence of elevated temperature to aid in the impregnation process. In some instances, depending on the constituents of the composition, it may be desirable to subject the textile, fabric or material to a subsequent step of washing or rinsing the textile, fabric or material with water to remove residual amounts of the composition which does not become impregnated in the textile, fabric or material. In some instances, depending on the constituents of the composition, it may be desirable to subject the textile, fabric or material to a subsequent step of exerting pressure on the textile, fabric or material to enhance the degree of impregnation and/or to aid in the removal of residual amounts of the composition or the solvent in which the composition is dissolved.

Other appropriate forms of treating textile, fabric or material with the compositions of the present disclosure include spraying, painting or misting the textile, fabric or material with the composition.

The following Examples provide further illustration of the various aspects of the embodiments of the disclosure described herein.

### Example I

A composition containing nonanal, decanal, (E)-2-hexenal, (E)-2-octenal, (2E,4E)-octadienal, benzaldehyde, (+)- and (−)-limonene, sulcatone, and benzyl alcohol is formulated to produce aggregation of bed bugs within an effective range of greater than 30 centimeters.

The composition made in accordance with Example I is contacted with a fiber textile, such as polyester woven material, by immersing a piece of the textile in a vat containing the composition. The fabric is allowed to remain in the vat of fluid for approximately one hour at room temperature. Thereafter, the fabric is removed from the vat and is allowed to air dry. The treated material is then processed as described further below.

### Example II

A composition for attracting bed bugs was formulated with (E)-2-hexenal in a concentration of less than 6×10¹⁵ molecules/ml (1.0×10⁻¹¹ M). Materials are then treated with the composition.

### Example III

A composition for attracting bed bugs is formulated with (E)-2-octenal in a concentration of less than 9×10¹⁴ molecules/ml (1.5×10⁻¹² M). Materials are then treated with the composition.

### Example IV

A composition containing a blend of volatile and low volatile attractant pheromones, such as those in feces and cuticular secretions, aggregation pheromones and alarm pheromones, from a list of pheromones described herein, is provided with an effective range, most generally, of 122 cm. The composition is produced by providing a rolled or folded piece of filter paper that has been exposed to and conditioned by bed bugs, as described previously herein. The paper is conditioned by exposure to the secretions and feces of the bed bugs. The conditioned paper is then installed within a perforate housing or delivery system, such as a pervious envelope or hollow tubule fitted with perforations for the emission of the volatiles to attract bed bugs to the insecticide-treated article. Notably, the delivery device may also be attached to or placed in proximity to the insecticide treated article.

Two experiments were conducted to demonstrate that bed bugs would move toward a potentiating agent, as described herein, and if the potentiating agent were associated with an insecticide, the bed bugs would die.

### Example V

A composition for treating an object is prepared combining from 100 mg/m² to 400 mg/m² of permethrin with from 5 mg/m² to 15 mg/m² of acetamiprid. The composition is then applied to an object, such as a mattress liner, by soaking the mattress liner in the composition and then air drying the mattress liner. A potentiating agent, such as (E)-2-hexenal, is placed (i.e., treated) on a carrier device, such as a 5 cm×10 cm piece of textile, and the carrier device is positioned in a perforate enclosure, which is then attached to the insecticide-treated object (e.g., mattress liner). Typically, permethrin as a synthetic pyrethroid, is impregnated at 550 mg/m², and in a range between 400 and 1250 mg/m². Acetamiprid, a neonicotinoid, is a pyrethroid having synergistic attributes, and may typically be applied at 19 mg/m². By utilizing a potentiator, it is possible to achieve the same level of insecticidal activity expressed by permethrin (100 to 400 mg/m²) and acetamiprid (5-15 mg/m²), individually, by attracting and having the bed bug remain resident on the surface for a longer period of time. This results in a more biocompatible liner for human use.

### Example VI

A composition for treating material or objects with an insecticide and a potentiating agent in admixture is prepared by combining permethrin in an amount of 550 mg/m² with octenal, an alarm pheromone, in an amount of 1.5×10⁻¹² M. The two compounds are mixed together and are then applied to a material or object, such as a mattress liner, by spraying the mattress liner with the admixture and allowing the mattress liner to air dry. At the stated low concentration, octenal behaves as a bed bug attractant, but will not be olfactorily detected by humans in close proximity

### Experiment 1

Test arenas were constructed using clear plastic tubes that were each 122 cm long by 10 cm in diameter. One end of each tube contained 15 cm of insecticide (permethrin) treated fabric. The remainder of the tube (117 cm) contained untreated fabric. A paper tent (4 cm square) that was conditioned by exposure to bed bug secretions and feces, and which was folded in half, was placed upon the treated fabric. Adult bed bugs were released into each tube from the end opposite the positioning of the conditioned paper tent. After two days, approximately 40% of the bed bugs were resting on the conditioned tents, compared to no bed bugs resting on unconditioned tents in control groups. Bed bugs traversed the whole length of the tube to seek harborage in the conditioned tents.

### Experiment 2

The same test arena was used as described in Experiment 1, and the same test methodology was also used, except that the conditioned paper tent was covered by insecticide (permethrin) treated fabric. After two days, approximately 25% of the bed bugs were resting on the conditioned paper covered with insecticide-treated fabric where approximately 25% mortality of those bed bugs had occurred, compared to the controls in which no bed bugs were observed resting on the insecticide-treated fabric and no mortality bed bug mortality was observed with the control tents that were not conditioned.

These experiments demonstrate that bed bugs can be attracted over long distances by the use of a conditioned paper acting as a potentiating agent, and that the attracted bed bugs will readily rest upon insecticide treated surfaces where bed bug mortality occurred.

The treated textiles, fabrics or materials of the present disclosure may be used in any number of consumer products, furnishings, fixtures and other uses. The treated textiles, fabrics or materials are particularly directed to use in products that may be exposed to insects, may typically become infested with insects or may be carrying means for transporting insects or their egg or larval forms from one site to another, thereby causing the spread of insect infestations.

For example, treated textiles, fabrics or materials may be used in the manufacture of commercial and residential flooring, such as carpets and rugs, upholstery for chairs, couches, benches and other items for seating, including but not limited to theater seats, airline seats, taxi seats, school bus seats, train seats or other public transportation seating.

The treated textiles, fabrics or materials may also be used to cover commercial or residential furnishings and fixtures such wall paper or textile wall coverings, and such as electrical features (e.g., switch plates and outlet covers), picture frames, headboards, box spring dust cover replacements, lamp shades and other items of furniture that may be covered in whole or in part with textiles or fabric.

The treated textiles, fabrics or materials may also be used for such items as pet beds and pet clothing. Treated textiles, fabrics or materials of the instant disclosure may be used in the manufacture of items of luggage, backpacks, tents, sleeping bags and other camping or outdoor equipment. The treated textiles, fabrics or materials may also be used in the manufacture of screening material for windows, doors, tents and conventional uses.

Treated textiles, fabrics or materials may also be used as backing material for any number of products that conventionally have a plastic or fluid impervious layer to provide a multilayer product having a plastic or fluid impervious layer with an insecticide-impregnated backing layer of textile or fabric material. Such items may include storage bags, purses, backpacks, garment bags for long-term storage of clothing, and the like.

The treated textiles, fabrics or materials of the present disclosure may be particularly suitable for use in manufacturing service items in the hotel/motel industry to prevent the spread of insect infestation, such as bed bugs. Such items may include, for example, bed linens, dust ruffles, box spring covers, floor coverings, such as carpets and rugs, curtains, wall coverings, lamp shades, electrical features, luggage rack slip covers and luggage rack cross-bracings (i.e., supportive straps) and other items typically found in hotel or motel rooms. Treated textiles, fabrics or materials may also be used in the manufacture of items used by the cleaning staff of hotels and motels, such as bags for collecting or transporting used and clean bed linens, towels, and other similar items, so that the transmission of insects or their eggs or larvae is limited or eliminated during the cleaning process.

The treated textiles, fabrics or materials may also be used in the manufacture of bags used in vacuum cleaners, laundry bags, suitcases, storage bags and other items where insects or insect infested items may collect or be transported.

The treated textiles, fabrics or materials may also be used in connection with outdoor furnishings, such as patio or picnic furniture, and screens for outdoor areas. Further, the treated textiles, fabrics or materials may be used in the manufacture of wrappings for agricultural or horticultural use, such a wraps for use in wrapping the trunks of trees or bushes to reduce insect infestations in plants, such as gypsy moth infestations.

The potentiating agents and compositions of insecticides and potentiating agents may be adapted for use in effectively killing any number of organisms that are known to infest or cohabitate on materials or articles that are commonly used by humans and animals. The descriptions of the various aspects of the embodiments of the disclosure are by way of example only, and not by way of limitation.

What is claimed is:

1. A composition for treating materials and articles used in human and animal habitation to provide reduction or elimination of insect infestation of the treated materials and articles, the composition containing an effective amount of at least one potentiating agent selected to provide an attractant shown to cause aggregation of insects toward a source of the attractant and a source of suitably effective insecticide to reduce or eliminate insect populations.

2. The composition of claim 1 wherein said at least one potentiating agent comprises one or more semiochemicals that are derived from natural sources.

3. The composition of claim 2 wherein said one or more semiochemicals is derived from secretions and feces of bed bugs.

4. The composition of claim 2 wherein said one or more semiochemicals comprise aggregation pheromones.

5. The composition of claim 2 wherein said one or more semiochemicals comprise alarm pheromones.

6. The composition of claim 1 wherein said at least one potentiating agent comprises one or more semiochemical-like compounds or compositions selected to attract insects to said at least one potentiating agent.

7. The composition of claim 2 wherein said at least one potentiating agent comprises one or more semiochemical-like compounds or compositions selected to attract insects to said at least one potentiating agent.

8. The composition of claim 1 further comprising one or more insecticidal agents in admixture with said at least one potentiating agent.

9. The composition of claim 8 wherein said one or more insecticidal agents are selected from the group comprising pyrethroids and neonicotinoids, and combinations thereof.

10. The compositions of claim 8 wherein said insecticidal agents are extracted from natural plant sources.

11. The composition of claim 1 wherein said composition is presented in a delivery system for placement on or in proximity to material or articles commonly used in human or animal habitation.

12. A delivery device for providing a potentiating agent selected to provide an attractant shown to cause aggregation of insects toward a source of the potentiating agent, the delivery device comprising a carrier device containing said potentiating agent, the carrier device being structured for attachment to or positioning in proximity to a material or article commonly used in human or animal habitation.

13. The delivery device of claim 12, further comprising an insecticide that is suitably effective to reduce or eliminate insect populations in or on said material or article commonly used in human or animal habitation.

14. The delivery device of claim 12, wherein said carrier device is a textile that has been treated or impregnated with said potentiating agent, said textile being sized to be received in a pervious enclosure.

15. A method of treating material or articles commonly used in human or animal habitation to reduce or eliminate insect infestation from the material or article, comprising:
providing a material or article;
providing a composition containing an effective amount of at least one potentiating agent selected to provide an attractant shown to cause aggregation of insects toward a source of the attractant; and
positioning said composition containing at least one potentiating agent on or in proximity to said material or article.

16. A method according to claim 15, wherein said composition further comprises one or more insecticidal agents in admixture with said at least one potentiating agent.

17. A method according to claim 15, further comprising treating said material or article with a composition comprising one or more insecticidal agents.

18. An article treated having insecticidal properties to reduce or eliminate insect populations in or on said article, comprising a material or article commonly used in human or animal habitation, said material or article being treated or impregnated with one or more suitable effective insecticidal agents in an amount sufficient to reduce or eliminate the number of insects in contact with said material or article, and further being treated with an effective amount of at least one potentiating agent selected to provide an attractant shown to cause aggregation of insects toward the potentiating agent and a source of said suitably effective insecticidal agent to reduce or eliminate insect populations in the material or article.

19. The article of claim 18, wherein said at least one potentiating agent is a semiochemical compound or semiochemical-like compound, or a combination thereof, shown to attract insects to the potentiating agent.

20. The article of claim 19, wherein said potentiating agent is derived from secretions and feces of bed bugs.
